(19) United States
(12) Patent Application Publication
(10) Pub. No.: US 2008/0142010 A1
(43) Pub. Date: Jun. 19, 2008

(54) SYSTEMS, METHODS, AND APPARATUSES FOR PULMONARY DRUG DELIVERY

(75) Inventors: Philip Weaver, Mouth of Wilson, VA (US); Lyndell Duvall, Fleetwood, NC (US); Jack Hebrank, Durham, NC (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NEXT SAFETY, INC., Jefferson, NC (US)

(21) Appl. No.: 11/950,180

(22) Filed: Dec. 4, 2007

Related U.S. Application Data
(63) Continuation-in-part of application No. 11/552,871, filed on Oct. 25, 2006.
(60) Provisional application No. 60/915,315, filed on May 1, 2007.
(60) Provisional application No. 60/826,271, filed on Sep. 20, 2006.

(51) Int. Cl. A61M 16/10; A61M 16/00; A61M 16/06

(52) U.S. Cl. 128/203.26; 128/203.12; 128/203.29

(57) ABSTRACT

In one embodiment, a system for pulmonary drug delivery includes a portable air supply unit comprising an air mover configured to generate a positive pressure airflow, a drug delivery unit configured to inject droplets of medication into the airflow generated by the air supply unit, and a user interface configured to deliver the airflow and droplets to a user.

### RELATED APPLICATIONS

This application claims the benefit of 60/915,315, entitled “Methods And Systems Of Delivering Medication Via Inhalation,” filed on May 1, 2007, and is a continuation-in-part of 11/552,871, entitled “Methods and Systems of Delivering Medication Via Inhalation,” filed on Oct. 25, 2006, which claims the benefit of 60/826,271, entitled “Methods And Systems Of Administering Medication Via Inhalation,” filed on Sep. 20, 2006. Each of those applications is hereby incorporated by reference into the present disclosure.

### BACKGROUND

The lung is the essential respiration organ in air-breathing vertebrates, including humans. Its principal function is to transport oxygen from the atmosphere into the bloodstream, and to excrete carbon dioxide from the bloodstream into the atmosphere. This exchange of gases is accomplished by a mosaic of specialized cells that form millions of tiny, thin-walled air sacs called alveoli. Beyond respiratory functions, the lungs also act as an efficient drug delivery mechanism. For example, the lungs have been used for centuries as a delivery mechanism for psychoactive drugs. One advantage of pulmonary drug delivery is that inhaled substances bypass the liver and the gastrointestinal tract and are therefore more readily absorbed into the bloodstream in comparison to orally-ingested medicines.

In recognition of the potential of pulmonary drug delivery, various efforts have been made toward developing effective pulmonary drug delivery devices. Current pulmonary drug delivery devices include metered dose inhalers (MDIs), dry powder inhalers (DPIs), and nebulizers. MDIs are pressurized hand-held devices that use propellants for delivering liquid medicines to the lungs. DPIs also use propellants, but deliver medicines in powder form. Nebulizers, also called “atomizers,” pump air or oxygen through a liquid medicine to create a vapor that is inhaled by the patient.

Each of the above-described devices suffer from disadvantages that decrease their attractiveness as a mechanism for pulmonary drug delivery. For example, when MDIs are used, medicine may be deposited at different levels of the pulmonary tree, and therefore may be absorbed to different degrees, depending on the timing of the delivery of the medicine in relation to the inhalation cycle. Accordingly, actual deposition of medicine in the lungs during patient use may differ from that measured in a controlled laboratory setting. Furthermore, a portion of the “metered dose” may be lost in the mouthpiece or the oropharynx.

Although DPIs reflect an effort to improve upon MDIs, small volume powder metering is not as precise as the metering of liquids. Therefore, the desired dosage of medicine may not actually be administered when a DPI is used. Furthermore, ambient environmental conditions, especially humidity, can adversely effect the likelihood of the medicine actually reaching the lungs.

Nebulizers may also exhibit unacceptable variability in delivered dosages, especially when they are of the inexpensive, imprecise variety that is common today. Although more expensive nebulizers are capable of delivering more precise dosages, the need for a compressed gas supply that significantly limits portability and the need for frequent cleaning to prevent bacterial colonization renders such nebulizers less desirable. Furthermore, the relatively high cost of such nebulizers also makes their use less attractive.

### BRIEF DESCRIPTION OF THE DRAWINGS

The disclosed systems, methods, and apparatus can be better understood with reference to the following drawings. The components in the drawings are not necessarily to scale, emphasis instead being placed upon clearly illustrating the principles of the present disclosure.

FIG. 1 illustrates an embodiment of a system for delivering drugs to the respiratory system under positive pressure.

FIG. 2 is a perspective view of an embodiment of a drug delivery unit used the system of FIG. 1.

FIG. 3 is an exploded perspective view of the drug delivery unit of FIG. 2.

FIG. 4 is a perspective view of an embodiment of a medicine containment element used in the drug delivery unit of FIGS. 2 and 3.

FIG. 5 is a cross-sectional view of the drug delivery device of FIG. 4.

FIG. 6 is a first cutaway partial view of an embodiment of an ejection head of the drug delivery unit of FIG. 4.

FIG. 7 is a second cutaway partial view of an embodiment of an ejection head of the drug delivery device of FIG. 4.

FIG. 8 is a cross-sectional view of the drug delivery device of FIG. 4.

FIG. 9 is a cross-sectional view of an alternative embodiment of a medicine containment element.

FIG. 10 is a perspective view of an alternative embodiment of a droplet ejection device.

FIG. 11 is a schematic view of an alternative embodiment of an ejection head.

FIG. 12 is a cross-sectional view of an alternative embodiment of a drug delivery unit.

FIG. 13 is a front view of an alternative embodiment of an air supply unit.

FIG. 14 is a partial cutaway, front view of a further alternative embodiment of an air supply unit.

FIG. 15 is a schematic view of a system for delivering medication to the respiratory system under positive pressure.

### DETAILED DESCRIPTION

### Pulmonary Drug Delivery

The present disclosure describes systems, methods, and apparatuses for delivering drugs (i.e., medicines, medications, pharmaceuticals, and other compounds) to the respiratory system. In some embodiments, the drugs are delivered at positive pressure. In further embodiments, the drugs are delivered with purified air.

There are several advantages to pulmonary drug delivery. For example, drugs delivered to the respiratory tract are not subject to complications with digestive tract chemistry. In addition, drugs absorbed by the lungs bypass the liver and are therefore not subject to first-pass metabolism as are orally delivered drugs. Pulmonary delivery is also non-invasive, requiring no needles or surgery. Moreover, the large surface area and sensitive nature of the membranes of the lungs provide a rapid and efficient means for delivering drugs into the bloodstream.

As described herein, drugs for pulmonary administration can be provided into a positive pressure (relative to atmospheric pressure) airstream that is delivered to a user (e.g., patient) during normal respiration. Such administration of drugs provides advantages beyond those associated with typical pulmonary drug delivery. For example, as will be apparent from the disclosure that follows, drugs can be continuously administered or administered in automatic coordination to the respiratory cycle of the user. Therefore, drugs can be delivered to the user in a highly controlled and targeted manner. In some embodiments, the drugs are administered with relatively low positive pressure airflows. That is, the airflows are lower in pressure than those provided by mechanical ventilators or continuous positive airway pressure (CPAP) machines. By way of example, the drugs are supplied in a gas, such as air, at a pressure of approximately 1 to 30 centimeters (cm) H₂O. Therefore, the drugs can be delivered to user without altering his or her normal breathing patterns.

As is further described in the following, the airflow can be purified prior to being provided to the user's respiratory tract. While the elimination of pollutants from the air can itself be considered a benefit to the user from the standpoint that environmental irritants of the lungs and other organs are reduced or eliminated, a closer examination of the composition of typical air, and particularly indoor air, reveals that purified air may be particularly important for ensuring effective and safe drug delivery via the pulmonary route. The importance of administration with purified air becomes apparent when the high concentrations and chemical composition of the particles normally found in environmental air are considered. While particle counts vary widely depending on the particular setting, indoor room air may easily contain greater than 10 billion particles per cubic meter, with many of those particles having diameters down to the 20 nanometer (nm) range. While there is a tendency to think of these particles as being inert objects, a large percentage of the particles are condensed droplets or micro-crystalline particles of organic and inorganic compounds, including such compounds as aromatic hydrocarbons and carbon particulates.

Further difficulties may arise due to the presence of ozone. While ozone is a harmful pollutant in it's own right, it is also highly reactive. Therefore, the reaction of ozone with other organically-based pollutants results in numerous derivative compounds that have been studied in some detail for outdoor air (the mechanisms of smog creation, etc.) but are not well documented in current literature and are not widely understood in indoor environments. Other organic compounds are also found in indoor air as a result of outgassing by polymers (carpet, upholstery, etc.) or simply as a result of the use of cleaning compounds. One class of organic compounds that have proven particularly active in forming derivative compounds in air when exposed to ozone are terpenes, which are used in many cleaners and air fresheners and which are responsible for the fresh pine or lemon scent of many cleaning products. Although many of these chemical reactions proceed relatively slowly, a high surface area-to-volume ratio increases the reaction rate between two compounds. With many aerosolized pollutant particles in the 20 nm range, the particles have a very large surface area to volume ratio resulting in rapidly occurring reactions.

An area of particular concern regarding the risk of undesirable chemical reactions between therapeutic drugs and environmental contaminants is the pulmonary delivery of proteins and peptides. As described in the review article by F. J. Kelly and I. S. Midway entitled “Protein Oxidation at the Air-Lung Interface,” Amino Acids 25: 375-396 (2003), which is hereby incorporated by reference into the present disclosure certain undesirable reactions are known to occur between proteins and reactive oxygen or nitrogen species such as ozone or nitrogen dioxide. As explained in greater detail in that article, reactive oxygen and nitrogen species and their secondary lipid and sugar oxidation products may interact with proteins causing reactions such as oxidation of the polypeptide backbone of the protein, peptide bond cleavage, protein-protein crosslinking, and a range of amino-acid side chain modifications. Both aromatic amino acids (e.g., tyrosine, tryptophan, phenylalanine) and aliphatic amino acids (e.g., arginine, lysine, proline, and histidine) may be targets of reactive oxygen and/or nitrogen species. Cysteine and methionine, the two sulphur-containing amino acids, appear especially sensitive to oxidation.

The combination of organic and inorganic pollutants with reactive chemistries, high particle counts, the presence of ozone, and uncertain derivatives as the result of ozone's interaction with other compounds make it difficult to predict air chemistry. Due to the possible formation of numerous compounds that would negatively impact the effectiveness of a drug being administered, or perhaps result in the creation of compounds that are detrimental to health, introduction of drugs into air that has not been adequately purified greatly increases the likelihood of negative effects. Hence, purified air is, at least in some embodiments, preferred for pulmonary drug delivery.

### EXAMPLE EMBODIMENTS

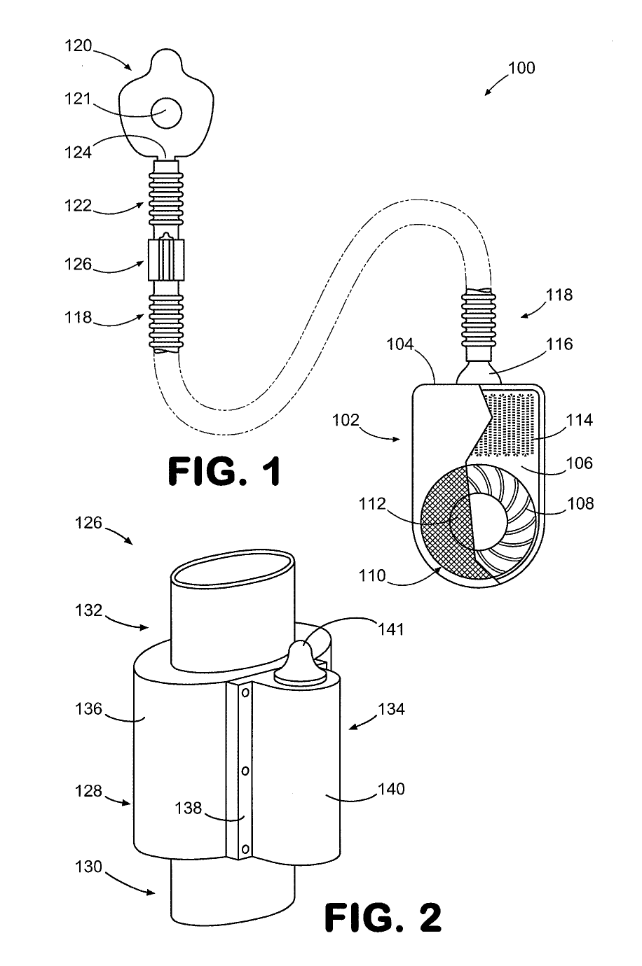

FIG. 1 illustrates an embodiment of a portable system for delivering drugs, such as medicines, to the respiratory system under positive pressure. As shown in FIG. 1, the system 100 comprises a portable purified air supply unit 102 that includes a housing 104 that defines an interior space 106. In at least some embodiments, the air supply unit 102 is portable in the sense that is small enough to be easily carried by the user, for example in one hand (in which case the unit may be considered to be a “handheld” unit), or worn by the user by being attached to the user's closing or strapped to the user's body, e.g., around the waist or over the shoulder, with an appropriate strap or leash. Provided within the interior space 106 is an air mover 108, such as a centrifugal blower or other fan, that is powered by an appropriate power source, such as a battery (not shown). The air mover 108 draws in air from the environment through an inlet 110 that, in some embodiments, includes a relatively coarse pre-filter 112 that filters relatively large particulate matter from the air before it reaches the interior space 106 of the housing 104. By way of example, the air mover 108 generates airflows of approximately 50 to 500 slm.

Also provided within the interior space 106 is a main particle filter 114 that is positioned downstream of the air mover 108. Air drawn into the housing 104 by the air mover 108 is forced through the main particle filter 114 such that nearly all of the particulate matter that remains in the air after it passes through the pre-filer 112 is retained in the main particle filter so as to purify the air. After passing through the main particle filter 114, the purified air is expelled from the housing 104 via an outlet 116.

With particle counts in environmental air at times measuring in excess of 10 billion per cubic meter in urban areas and with particle sizes down to 20 nm, careful consideration must be given to filtration. The standard for most consumer, occupational, and medical filtration devices is currently high efficiency particulate air (HEPA) grade filtration (99.97% efficiency at 300 nm). If such a filter were used, however, over 10 million particles would still pass through the filter for every cubic meter of air.

In order to ensure filtration at efficiencies that will substantially eliminate the potential for harmful reactants resulting from high concentrations of unknown airborne chemicals reacting with drugs, the system 100, in one embodiment, implements ultra-low penetration air (ULPA) filter material for the filter 114. Suitable ULPG grade filter materials are available from Lydall Filtration/Separation, Inc., Rochester, N.H. Although such filter material has been used in clean rooms, it has not been used in smaller applications for breathable air such as that described herein.

As depicted in FIG. 1, the filter 114 has an accordion configuration in which the filter is folded over on itself in alternate directions. Such a configuration increases the surface area of the filter 114. By way of example, the filter 114 has a usable surface area of approximately 2700 cm² to 5400 cm². Because, at a given flow rate, face velocity is inversely proportional to filter area, the surface area of the filter 114 is larger than that required to satisfy pressure drop requirements in order to establish very low particle velocities, thereby providing extremely high efficiencies that may be important for combining the drugs and the air. By further way of example, the filter 114 is configured to filter out particles having a size of 10 nm or more.

Existing respirators typically achieve a filtration efficiency of approximately 99.97% at 300 nm. With indoor air particle concentrations of about 10 billion particles per cubic meter and a pulmonary inspiration volume at rest of up to about 5 liters, such filtration allows passage of more than approximately 15 thousand particles per inspiration of sizes equal to 300 nm in diameter and more than 150 thousand at sizes of about 25 nm and smaller. The filter 114, however, is capable of filtration efficiency of approximately 99.99996% and approximately 99.99999% at 2700 cm² and 5400 cm², respectively, thereby limiting passage of particles to mere hundreds of particles per inspiration.

Although filtration of particulate matter in the manner described above provides a significant improvement, ozone, as a molecular level substance, can remain as a pollutant in the filtered air. Therefore, in some embodiments, ozone is also removed by a reaction or catalytic process in which the ozone is converted to molecular oxygen or into other compounds that are not harmful or that are less reactive than ozone. In some embodiments, the ozone can be reduced or eliminated through use of activated carbon. The activated carbon can, for example, be impregnated into the material of the filter 114. Alternatively, activated carbon, for example in granulated form, can be contained within the filter 114, for example held between two layers of filter material.

Given that the performance of activated carbon deteriorates over time and may need to be periodically replaced, a catalyst that assists in the conversion of ozone to oxygen can alternatively, or additionally, be used. Example catalysts include MnO₂ (both γ-MnO₂ and β-MnO₂), palladium or palladium oxides, and Ag₂O and other metal oxides such as aluminum oxides or copper oxides. In some embodiments, one or more such catalysts can be applied as a coating on interior surfaces of the system 100 that are in contact with the airstream, such as the interior surfaces of the housing 104 or supply hoses (described below) that deliver air to the user. Alternatively or additionally, one or more catalysts can be incorporated into the filter material, for example by impregnation or adhering particles of the catalyst(s) to the fiber matrix of the filter 114. In further embodiments, the catalyst(s) can be incorporated into the filter fibers themselves.

In cases in which MnO₂ is used as a catalyst, SO₂, which is another major air pollutant, can also be reduced or eliminated. Furthermore, NO₂, can be catalyzed using various chemicals in conjunction with some energy to drive a reaction. For example, photocatalysis of oxides of nitrogen may reduce or eliminate NO₂ when exposed to an irradiated surface of TiO₂.

With further reference to FIG. 1, the outlet 116 connects to a first supply hose 118, which is used to deliver the purified air toward the user. As indicated in FIG. 1, the outlet 116 can be fitted within the supply hose 118. In other embodiments, however, a reverse arrangement can be used in which the supply hose 118 is fitted within the outlet 116. To prevent leakage of air out from or into the system 100 at the connection point between the outlet 116 and the supply hose 118, either or both of the outlet and supply hose can be provided with one or more sealing members (not shown) that ensure a positive seal. In the embodiment of FIG. 1, the supply hose 118 comprises a ribbed hose or tube. It will be appreciated, however, that many other configurations are possible and may be used with similar results in the system 100.

The system 100 further includes a user interface 120 that may be donned by the user or otherwise positioned so as to enable the delivery of purified air and medication to the user's respiratory tract via the nose and/or mouth. As shown in FIG. 1, the user interface 120 can, for example, comprise a face mask. In some embodiments, the user interface 120 includes a pressure-relief valve 121 that is used to release air exhaled by the user and/or air supplied by the supply unit 102, for example during instances of user exhalation during the respiratory cycle. The user interface 120 is connected to a second supply hose 122, which delivers air to an inlet 124 of the user interface. The user interface inlet 124 can fit within the supply hose 122 or a reverse arrangement can be used. Regardless, either or both of the inlet 124 and the supply hose 122 can comprise one or more sealing members (not shown) to provide a positive seal between the inlet and the supply hose. In the embodiment of FIG. 1, the supply hose 122 also comprises a ribbed hose or tube. It will be appreciated, however, that many other configurations are possible and may be used with similar results in the system 100.

In addition to the above-described components, the system 100 also comprises a drug delivery unit 126 that is used to add one or more drugs to the purified air that are to be delivered to the user. The drug delivery unit 126 is connected to both the first and second supply hoses 118 and 122. In some embodiments, portions of the drug delivery unit 126 are received within the supply hoses 118, 122. In other embodiments, the supply hoses 118, 122 are received within the drug delivery unit 126. One or more sealing members (not shown) can be provided on either or both of the drug delivery unit 126 and the supply hoses 118, 122 to ensure a positive seal and prevent the ingress or egress of air at the connection points between the medical port and the supply hoses. An example embodiment of the drug delivery unit 126 is described in relation to FIGS. 2-8 in the following.

With reference next to FIG. 2, the drug delivery unit 126 is illustrated separate from the remainder of the system 100. As indicated in FIG. 2, the drug delivery unit 126 generally comprises a body portion 128 from which extend an inlet 130 and an outlet 132. In the embodiment shown in FIG. 2, each of the inlet 130 and outlet 132 comprise a short, hollow tube having a generally cylindrical shape. Although a cylindrical shape has been described, substantially any other shape can be used as long as a positive seal is made between the drug delivery unit 126 and the first and second supply hoses 118, 122 (FIG. 1). As is further indicated in FIG. 2, the body portion 128 is larger in circumference than the inlet 130 and outlet 132, but it need not necessarily be so. In some embodiments, however, the relatively large size of the body portion 128 facilitates the mounting of additional components on or within the drug delivery unit 126. In the embodiment shown in FIG. 2, the body portion 128 is generally cylindrical although, again, substantially any other shape could be used.

As indicated in FIG. 2, a medication containment element 134 is attached to the body portion 128. More particularly, the medication containment element 134 is attached to the body portion 128 at a point along the body portion's outer periphery 136, for example at a position in which the containment element faces outward from the user when the system 100 is being used (see orientation of FIG. 1). The medication containment element 134 can be attached to the body portion 128 in any number of ways. By way of example, the medication containment element 134 can be attached by gluing or otherwise bonding, or through use of mechanical fasteners, such as screws. As is apparent from FIG. 2, the medication containment element 134 includes a mounting flange 138 that is used to attach or mount the medication containment element to the body portion 128, and a container 140 that is used to hold medication in solution form that is to be added to the stream of purified air that is delivered to the user. In addition, the medication containment element 134 can include a cap 141 or other closure member that is used to seal the container 140 after a desired amount of medication has been added to the container.

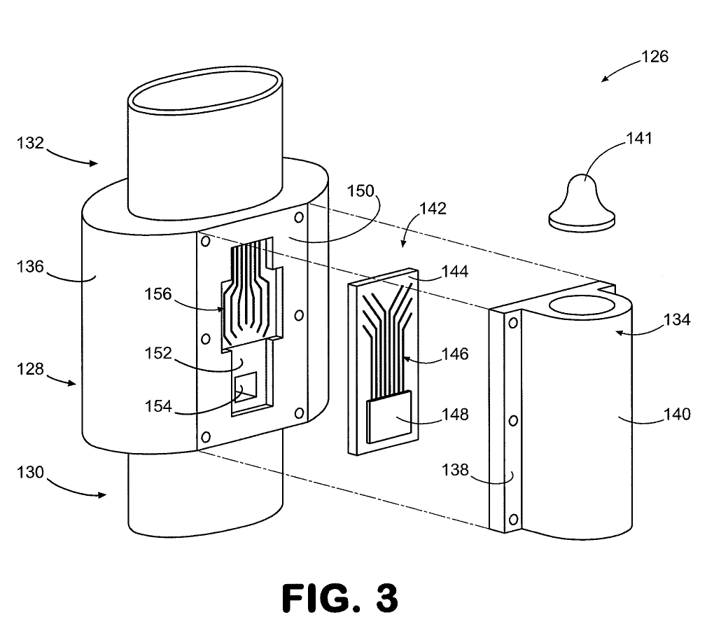

FIG. 3 shows the drug delivery unit 126 in a partially-exploded view with the medication containment element 134 separated from the body portion 128. As indicated in FIG. 3, a droplet ejection device 142 is positioned between the medication containment element 134 and the body portion 128. As described in greater detail below, the droplet ejection device 142 is used to selectively eject fine droplets of medication into the stream of purified air that flows through an internal passage of the drug delivery unit 126. The droplet ejection device 142 generally comprises a substrate 144 on which various conductor traces 146 are formed that connect with droplet ejection elements (not visible in FIG. 3) that individually eject the droplets of medication. In the embodiment of FIG. 3, the droplet ejection elements are provided within an ejection head 148 of the droplet ejection device 142 at a bottom end (in the orientation shown in FIG. 3) of the substrate 144.

In the embodiment of FIG. 3, the body portion 128 includes a generally planar mounting surface 150 to which the medication containment element 134 can attach. A trench or cavity 152 is formed in the mounting surface 150 that is shaped and configured at least to receive the droplet ejection device 142 such that the droplet ejection device is substantially flush with the mounting surface when inserted into the cavity. In such a case, the cavity 152 can have a depth that is similar in dimension to the thickness of the droplet ejection device 142. The cavity 152 further includes an injection port 154 through which ejected droplets of medication can pass, and additional conductor traces 156 that are adapted to connect with contacts (not shown) provided on the substrate 144. The additional conductor traces 156 can, in some embodiments, comprise part of a ribbon cable or other element (not shown) with which connectivity between the droplet ejection device 142 and a control unit (described later) can be facilitated.

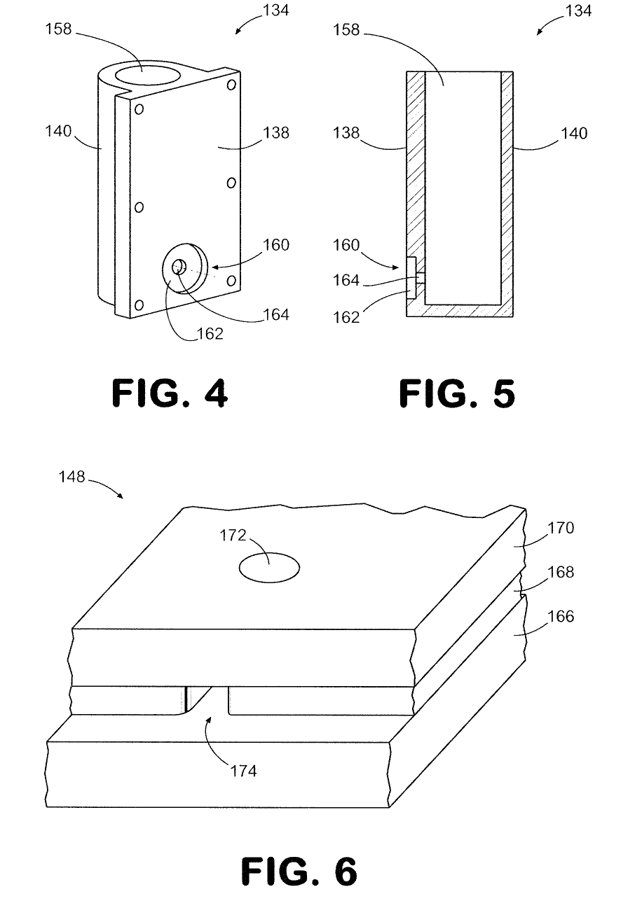

FIGS. 4 and 5 illustrate an example embodiment of the medication containment element 134. Beginning with FIG. 4, the container 140 of the medication containment element 134 defines an inner reservoir 158 that is used to hold medication for introduction into the purified airstream. As indicated in FIG. 4, the reservoir 158 can be generally cylindrical, although other shapes are possible. Also shown in FIG. 4 is an outlet port 160 through which the contained medication is supplied to the droplet ejection device 142 (FIG. 3). In the embodiment of FIG. 4, the outlet port 160 comprises a relatively large first bore 162 that surrounds or contains a relatively small second bore 164. With reference to FIG. 5, which comprises a cross-sectional view of the medication containment element 134, the second bore 164 is in fluid communication with the reservoir 158. Therefore, medication contained in the reservoir 158 can pass, due to gravitational forces and/or due to capillary action (described below), through the second bore 164, through the first bore 162, and to the droplet ejection device 142.

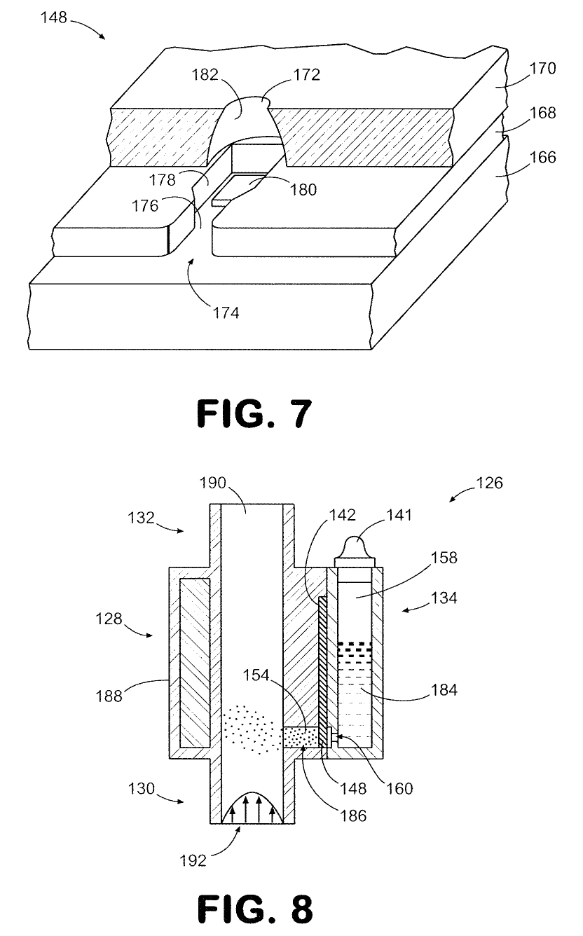

FIGS. 6 and 7 illustrate an example configuration for the ejection head 148 of the droplet ejection device 142 shown in FIG. 3. More particularly, FIGS. 6 and 7 illustrate a cutaway portion of an embodiment of the ejection head 148. Beginning with FIG. 6, the illustrated embodiment of the ejection head 148 includes multiple layers of material, including a first layer 166, a second layer 168, and a third layer 170. In some embodiments, the first layer 166 comprises a substrate, the second layer 168 comprises barrier layer, and the third layer 170 comprises an orifice plate. For purposes of the following discussion, it is assumed that the layers 166, 168, and 170 respectively comprise a substrate, a barrier layer, and an orifice plate.

The substrate 166 provides a support or base for the ejection head 148. In some embodiments, the substrate 166 is formed from a semiconductor material, such as silicon. The barrier layer 168 is provided on top of the substrate 166 and insulates conductive traces (not shown) of the substrate from the remainder of the ejection head 148. In some embodiments, the barrier layer 168 is formed from a non-conductive material, such as a polymer. The orifice plate 170 includes nozzle orifices 172 from which droplets of medicine are ejected during use of the ejection head 148. In some embodiments, the orifice plate 170 is formed from a metal material.

With further reference to FIG. 6, a supply inlet 174 is formed in the barrier layer 168 and defines a pathway for medicine to flow prior to being ejected from the ejection head 148. Turning to FIG. 7, which shows the cutaway portion of FIG. 6 with a further portion of the orifice plate 170 removed (indicated by crosshatching), the supply inlet 174 opens to a supply channel 176 that feeds medicine to a firing chamber 178. Provided within the firing chamber 178, and for example formed on the substrate 166 within the firing chamber, is an droplet ejection element 180, which is responsible for driving medicine through a nozzle 182 formed in the orifice plate 170 and out from the nozzle orifice 172. In some embodiments, the droplet ejection element 180 comprises a heater resistor, such as a thin-film heater resistor. Other configurations for the droplet ejection element 180 are, however, possible. For example, the droplet ejection element 180 can alternatively comprise a piezoelectric pump element.

During use, medicine, in liquid form, is delivered via the inlet 174 and the channel 176 to the firing chamber 178. When it is desired to eject medicine from the ejection head 148 using the droplet ejection element 180, the droplet ejection element is energized, for example using the aforementioned substrate conductor traces. In embodiments in which the droplet ejection element 180 comprises a heater resistor, a thin layer of the medicine within the firing chamber 178 is superheated, causing explosive vaporization and ejection of a droplet of medicine through the nozzle 182 and orifice 172. Ejection of the droplet then creates a capillary action that draws further medicine within the firing chamber 178 such that the ejection head 148 can be repeatedly fired. Using the ejection head 148, the sizes of the ejected droplets can be reproduced with great precision. For example, in some embodiments, the ejection head 148 can eject droplets approximately half of which being within approximately 500 nm of each other in terms of diameter.

Turning to FIG. 8, an embodiment of use of the drug delivery unit 126 will be described. As indicated in FIG. 8, the inner reservoir 158 of the medicine containment element 134 is at least partially filled with an amount of medicine 184. The medicine can comprise substantially any compound that is to be delivered to the respiratory tract of the user. Once provided in the reservoir 158, the medicine can flow through the outlet port 160 to the droplet ejection device 142 and, more particularly, to the ejection head 148 of the droplet ejection device. The medicine can then fill the various firing chambers (e.g., chamber 178 in FIG. 7) of the ejection head 148 and the droplet ejection elements (e.g., element 180) provided within or adjacent the chambers can be energized to eject droplets 186 of the medicine into the injection port 154 of the body portion 128. In some embodiments, the droplet ejection elements are energized under the control of a control unit 188 provided on or within the drug delivery unit 126. Irrespective of its location, the control unit 188 can comprise a programmable, integrated logic circuit that includes one or more of a processor, memory, and a power supply (e.g., battery). Within memory are stored various routines or programs that can be used to control operation of the drug delivery unit 126 and its components, such as the droplet ejection device 142. In some embodiments, the control unit 188 can control how much medicine is administered, for example by controlling how often medicine can be ejected and for how long (i.e., at what dosage). For example, when a pressure sensor is provided in the system, for instance within the user interface 120 (FIG. 1) or the drug delivery device 126, the control unit can activate the droplet ejection device 142 when a pressure drop indicative of user inhalation is detected and reported by the pressure sensor.

The droplets 186 are ejected with sufficient force and velocity to propel them through the injection port 154 and into an inner passage 190 of the drug delivery unit 126 so as to be positioned to be carried toward the user's respiratory tract by a stream of purified air 192 generated by the purified air supply unit 102 (FIG. 1).

### Droplet Size Control

In order to achieve effective systemic absorption of drugs delivered by the respiratory tract, it is normally desirable to deliver the drug directly to the alveoli located deep within the lung structure where transport to the bloodstream is quickly and efficiently accomplished. The processes of impaction, sedimentation, and diffusion each plays a role in determining where airborne particles are ultimately deposited within the lung. Impaction is the tendency of particles to maintain a path despite changes in the direction of the airstream and is a primary factor involved in the deposition of large particles (diameters of 10 microns (μm) or larger) in the upper airways. Sedimentation, which is the process by which particles “settle out” due to gravity, and diffusion, which is the process by which particles contact the walls of airways due to random motion, play increasing roles deeper in the lung and for smaller particle sizes (diameters less than 10 μMm).

Lung deposition curves, such as those published by the International Commission on Radiological Protection (ICRP), indicate that the locations within the pulmonary tree in which inhaled particles are deposited also depends to a substantial degree upon particle size. Specifically, lung deposition curves based on both theoretical modeling and experimental data typically show that particle deposition rates in the alveolar regions of the lung are greatest for particles having an aerodynamic diameter of approximately 1 to 3 μm.

In view of such data, it would appear prudent to generate medication droplets having a diameter in the 1 to 3 μm range. Therefore, in some embodiments, the droplet ejection device described above and used to eject medication can be configured to eject droplets having diameters in that range. Generally speaking, the diameter of an ejected droplet will be about the same as the diameter of the orifice from which the droplet was ejected. Therefore, by way of example, a droplet ejection device having nozzle orifices with 20 μm diameters can, under typical use conditions, be expected to eject droplets having diameters around 20 μm.

The potential benefits of smaller nozzle orifices have been recognized. For example, in the inkjet printing arts, it has been recognized that smaller orifices may translate into higher printing resolution. Accordingly, attempts have been made to create droplet ejection devices, such as inkjet printheads, having orifices smaller than 10 μm. Unfortunately, there are impediments to creating droplet ejection devices having orifices of such small dimensions. First, very precise manufacturing techniques are required to enable repeatable formation of components comprising orifices of very small diameters. Second, even when such techniques are successfully performed, effective and controlled droplet ejection can be difficult to achieve due to the physics involved when ejecting a liquid from such a small orifice. For example, as the orifice size decreases, the surface tension and viscosity force imposed upon the liquid increase, thereby requiring higher actuation pressures to eject droplets. At least in part due to one or more those reasons, current inkjet printheads typically comprise orifices in the range of approximately 15 to 30 μm. Indeed, printheads with 15 μm diameter orifices are considered to be state-of-the-art printheads.

In view of the above, it would be desirable to have a way to decrease droplet size without having to further reduce orifice sizes. As described in the following, various other factors or parameters can be manipulated to control the size of the droplets (i.e., particles) that are provided to the respiratory tract. Through such manipulation, droplets of a desired diameter, such as approximately 1 to 3 μm, can be delivered to the alveoli to obtain desired deposition and absorption.

Generally speaking, the size of the droplets can be controlled during droplet formation, after droplet formation, or both. During droplet formation, certain parameters can be controlled to alter the size of the droplets that are ejected. In some cases, the droplet size may not necessarily be the same as the size of the nozzle orifice. For example, droplets that are smaller than the nozzle orifice may be produced. After droplet formation, certain other parameters can be controlled to change the size of the generated droplets. For example, the droplets can be reduced in size downstream of the nozzle orifice through controlled evaporation. Using such processes, an droplet ejection device having relatively large (e.g., approximately 10 to 30 μm) orifices can still be used to deliver substantially smaller (e.g., approximately 1 to 3 μm) droplets to the alveoli.

Regarding droplet formation, it has been determined that relatively small droplets can be generated when the liquid from which the droplets are formed is maintained at an elevated temperature. Such elevated temperatures decrease both the viscosity and surface tension of the liquid, which translates into smaller droplets being ejected. In some embodiments, liquid temperatures in the range of approximately 45 to 110° C. are effective in reducing droplet diameter, with temperatures of approximately 90 to 99° C. being preferred in some embodiments. Notably, the composition of the liquid (e.g., medication solution) can also affect droplet size. Therefore, results may vary depending upon the nature of the medication being administered. Furthermore, relatively high droplet temperatures may increase droplet evaporation that, as described below, can significantly reduce the size of the droplets.

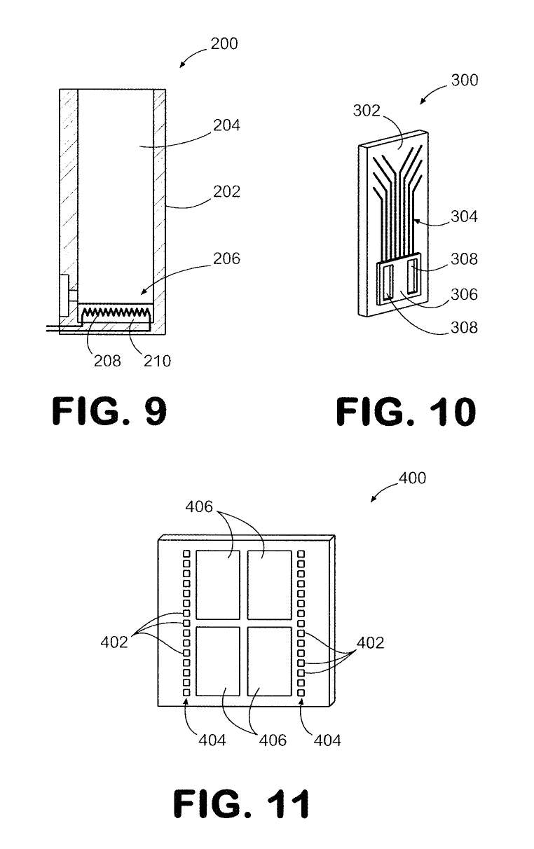

Medication used in the system 100 can be heated using a variety of methods. Generally speaking, any method with which the medication is heated prior to its ejection (i.e., preheated) can be used. FIG. 9 illustrates a first preheating implementation. As indicated in FIG. 9, a medication containment element 200 similar to the element 126 described in the foregoing is provided. Therefore, the medication containment element 200 includes a container 202 that defines an inner reservoir 204. In the embodiment of FIG. 200, however, a medication heating element 206 is provided in the bottom of the reservoir 204. By way of example, the heating element 206 comprises a resistance heater that includes a heating coil 208 that is contained or encapsulated within a thermally-conductive member 210.

FIG. 10 illustrates a second preheating implementation. As indicated in FIG. 10, a droplet ejection device 300 similar to that described in the foregoing is provided. Therefore, the droplet ejection device 300 includes a substrate 302, conductive traces 304, and an ejection head, which is not visible in FIG. 10. In the embodiment of FIG. 10, however, a medication heating element 306 is provided in close proximity, and in some embodiments on top of, the ejection head. In cases in which the heating element 306 contacts the ejection head, the heating element may be designated as an ejection head heating element and may be energized using one or more of the traces 304. By way of example, the heating element 306 comprises a resistance heater similar in nature to the heating element 206 used in the reservoir 204 (FIG. 9). As is further illustrated in FIG. 10, the heating element 306 may comprise grooves or slots 308 that enable the medication supplied by a medication containment element to reach the droplet ejection elements of the ejection head.

In a third preheating implementation, one or more heater resistors of the ejection head can be used to heat the medication prior to its ejection. For example, some of the heater resistors can be utilized as designated preheaters, or each of the various heater resistors that are used for droplet ejection can be configured to first preheat the medicine contained within the firing chambers with relatively low energy, and then eject the medicine as a droplet with high energy once a desired temperature has been reached. Alternatively, additional heater resistors can be provided, for example between aligned rows of resistor heaters. Such an arrangement is schematically depicted in FIG. 11 for an ejection head 400. The ejection head 400 comprises a plurality of heater resistors (or other ejection elements) 402 that are provided in rows 404. Between the rows 404 are multiple heater elements 406, which can be energized to heat the medicine before ejection.

As mentioned above, droplet size can be controlled after formation. The exercise of such control may generally be referred to as post-processing of the droplets. It has been determined that droplet size can be significantly reduced due to evaporation of the ejected droplets during their flight to the user's respiratory tract. Such evaporation may occur naturally as a consequence of the current environmental conditions in which the system is used, such as temperature, humidity, and pressure. As the droplets evaporate, they lose fluid (e.g., water), which results in a corresponding loss of mass and volume and, ultimately, droplet diameter. Discussed in the following are several factors or parameters that affect droplet evaporation rate and which therefore can be used to control (e.g., decrease) droplet size.

One factor or parameter that has a significant impact on droplet evaporation and that can be controlled is air temperature. Specifically, the higher the temperature of the air that is being used to deliver the droplets to the respiratory tract, the greater the evaporation rate. Therefore, droplet size can be reduced by heating the air that flows through the system. In some embodiments, the air is heated from an ambient temperature (e.g., room temperature) to a temperature of approximately 20 to 60° C. The extent of droplet evaporation and size reduction obtained is dependent upon the particular air temperature that is reached as well as the duration of time the droplets are present within the heated air (i.e., time of flight to the respiratory tract), with higher temperatures and longer times of flight resulting in greater evaporation. The time of flight corresponds to the distance the droplets must travel to reach the respiratory tract and the speed with which the air is flowing toward the user. Therefore, the temperature to which the air is heated, the position at which the drug delivery unit is located relative to the patient interface, and the speed setting for the air supply blower can each be selected to obtain desired evaporation results.

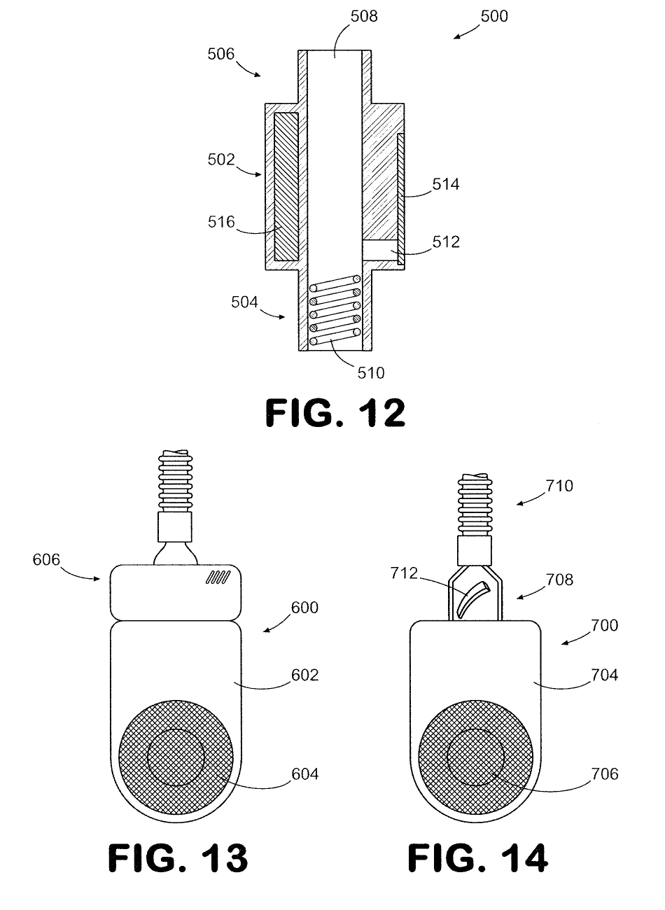

FIG. 12 illustrates a drug delivery unit 500 with which air can be heated to control droplet evaporation. The drug delivery unit 500 is similar to that described above and therefore comprises a body portion 502, an inlet 504, and an outlet 506, which together define an inner passage 508. In the embodiment of FIG. 12, however, an air heating element 510 is provided within the inner passage 508 at the inlet port 512. The heating element 510 can comprise a resistance heater that includes a coil or other configuration of resistive material that generates heat when energized. In some embodiments, the heating element 510 can be powered by a control unit 516. In other embodiments, the heating element 510 can be powered by the air supply unit 102 (FIG. 1). Although the heating element 510 is shown in FIG. 12 as being provided within the inner passage 508, the heating element alternatively could be integrated into the structure (e.g., walls) of the inlet 504 so as to avoid disruption of airflow through the inner passage. As described below, however, such disruption may be desirable given that turbulence may also be used to alter the size of droplets.

In a further embodiment, droplets flowing through the system can be heated using photon absorption. For example, a light source, such as an infrared light source, can emit photons from within the drug delivery unit or supply hose that become absorbed by the droplets. In some embodiments, the inner surfaces of the drug delivery unit and/or supply hose can be coated with a reflective material (e.g., a dielectric stack) that reflects the photons, potentially multiple times, to increase the chances of the photons being absorbed by droplets.

Another factor or parameter that has a significant effect on droplet evaporation that can be controlled is the relative humidity of the air used to carry the droplets to the user. As one would expect, the lower the relative humidity of the air, the greater the droplet evaporation rate and therefore the smaller the diameter of the droplets when they reach the respiratory tract. In some embodiments, the air is dehumidified from an initial relative humidity (e.g., 60%) to a reduced relative humidity of approximately 50% or less. The extent of droplet evaporation and size reduction that can be achieved is dependent upon the particular environmental relative humidity and the duration of time the droplets are present within the airstream (time of flight), which corresponds to both the distance the droplets must travel to reach the respiratory tract and the speed with which the air that carries the droplets is flowing. Therefore, the relative humidity to which the air is reduced, the position at which the drug delivery unit is located relative to the patient interface, and the speed setting for the air supply blower can each be selected to obtain desired evaporation results. Notably, although dehumidification has been described as a means to decrease the size of the medicine droplets, it is noted that humidification could alternatively or additionally be used to increase the size of the medicine droplets, if desired. For example, the relative humidity of the air can be reduced to a significant extent just upstream from the drug delivery unit, for example to near 0% humidity, and then increased significantly just prior to the air entering the respiratory tract. In such a case, substantial droplet size reduction can be achieved without providing undesirably dry air to the user.

FIG. 13 illustrates an air supply unit embodiment with which air can be humidified and/or dehumidified to control droplet evaporation. The air supply unit 600 is similar to that described above, and therefore comprises a housing 602 having an inlet 604. In the embodiment of FIG. 13, however, the unit 600 includes a conditioning unit 606 that can be used to reduce and/or increase the relative humidity of air expelled by the unit's blower. In terms of dehumidification, the conditioning unit 606 can comprise one or more of desiccant material and a condenser. In terms of humidification, the conditioning unit 606 can comprise one or more of a vaporizer, nebulizer, or other atomizer configured to vaporize a liquid (e.g., water) into a gaseous form. In other embodiments, humidification can be provided with a containment element and droplet ejection mechanism similar to those used to provide medication to the airstream.

A further factor or parameter that has a significant effect on droplet evaporation is the turbulence of the airstream used to carry the droplets to the user. Generally speaking, the higher the turbulence, the greater the evaporation rate and therefore the smaller the diameter of the droplets when they reach the respiratory tract. In some embodiments, turbulence can be created by adding one or more turbulence creation members along the flow path from the air supply unit to the patient interface. In some embodiments, such turbulence creation members can comprise static or moving (e.g., spinning) vortex generators. In some embodiments, the Reynold's number of the airflow can be at least 3,000 to obtain effective droplet evaporation.

FIG. 14, illustrates a further air supply unit 700 comprising a housing 704, an inlet 706, and an outlet 708 that is connected to a delivery hose 710. A turbulence creation member 712 in the form of a static fin is shown formed within the outlet 708. The extent of droplet evaporation or size reduction that can be achieved is dependent upon the degree of turbulence that is achieved and the duration of time a given droplet is present within the airstream, which may correspond to both the distance the droplets must travel to reach the respiratory tract and the speed with which the air that carries the droplets is flowing. Therefore, the turbulence of the airflow within the supply hoses, the position at which the drug delivery unit is located relative to the patient interface, and the speed setting for the air supply blower can be each selected to obtain desired evaporation results.

Yet another factor or parameter that has a significant effect on droplet evaporation is the composition of the droplet. In particular, the nature of the solution used to form the droplets can have a significant effect on the rate at which the droplets evaporate. The evaporation rate of droplets depends to a significant extent on the properties of the solvent and the solutes present within the solvent. Volatile liquids (i.e., those with relatively high vapor pressures) evaporate more quickly than non-volatile liquids. Various solutes tend to affect the vapor pressure of the droplet surface in particular ways. Saline solutions, which comprise water and sodium chloride, are widely used as carriers for medicinal compounds due to their similarity to and compatibility with human tissues and biological processes. The evaporation of water is well understood. The presence of sodium chloride, however, tends to lower vapor pressures.

Evaporation and condensation typically occur simultaneously at the air-liquid interface of liquid droplets. The ratio of evaporation rate to condensation rate is dependent upon the vapor pressure at the droplet surface. As the concentration of sodium chloride in a saline solution increases, the ratio of evaporation to condensation decreases. At low relative humidity and elevated temperatures, saline solutions (e.g., a 0.9% solution) tend to have evaporation rates that are higher than condensation rates with a net result of evaporation and droplet shrinkage. As relative humidity increases (as in the respiratory tract), the rate of condensation relative to evaporation becomes larger until the droplet begins to gain mass and increase in size. Increasing the solute concentration in such a case will shift the point at which evaporation and condensation are at equilibrium to a point of lower humidity and higher temperature.

In the foregoing, various factors or parameters have been described that affect droplet evaporation and which therefore can be manipulated to control droplet size. Although each parameter is discussed separately, two or more of the parameters can be individually or simultaneously controlled in order to achieve a desired degree of evaporation and therefore a desired droplet size. Indeed, in some embodiments, each of the air temperature, air relative humidity, airflow turbulence, and droplet composition can be controlled to achieve optimum droplet evaporation.

Furthermore, it will be appreciated that current operating conditions may have an effect on droplet evaporation or on the operation of components that control droplet evaporation (e.g., resistance heaters, dehumidifiers, etc.). For example, if the system is being used in a relatively dry environment, further dehumidification may be unnecessary. In such a case, any dehumidification components provided in the system can, at least temporarily, be deactivated. Stated in the alternative, the dehumidification component(s), when provided, can be selectively activated when necessary based upon sensed ambient conditions. The same form of control may apply in cases in which the system is used in relatively hot environments. In such a case, the air may not need to be heated to the same extent as would be necessary when the system is operated in colder conditions. As a further example, if the operating environment is very dry, it may be determined that not only is dehumidification unnecessary, but air heating is also unnecessary.

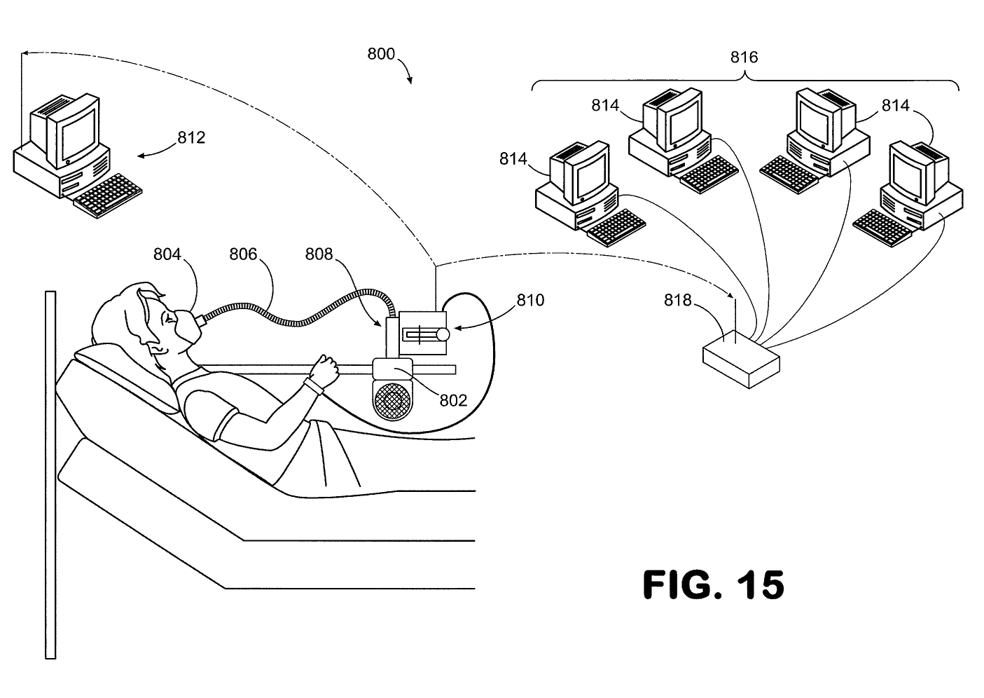

FIG. 15 illustrates a further embodiment of a system 800 for delivering drugs to the respiratory system under positive pressure. As indicated in FIG. 15, the system 800 comprises a purified air supply unit 802 and a user interface 804 that are connected by a supply hose 806. Provided along the length of the supply hose 806 is a drug delivery unit 808 that can comprise control features described above. Connected to the drug delivery unit 808 is a monitoring unit 810 that collects patient data, such as blood pressure, heart rate, blood oxygen saturation, or blood glucose levels. In addition, the monitoring unit 810 can collect data from the drug delivery unit 808, such as measured respiration rates and respiratory volume. Such data can then be transmitted by the monitoring unit 810 to another component, such as a local computer 812, either via a wired or wireless connection. Furthermore, the data can be transmitted to one or more remote computers 814 also via a wired or wireless connection. By way of example, the remote computers 814 can comprise part of a remote local area network (LAN) 816 that is wirelessly connected to the monitoring unit 810 with a wireless node 818. When wireless communications are used, one or more wireless protocols such as one or more broadband protocols, IEEE 802.11, Bluetooth, or Zigbee may be used.

With the arrangement shown in FIG. 15, it is possible for a health care professional such as a nurse or physician to both monitor conditions of the patient remotely and control the drug delivery unit 808 to adjust dosage, frequency of delivery, temperature, humidity, etc. of the airflow to the patient from a remote location relative to those conditions.

### Further Embodiments

While particular embodiments of systems, methods, and apparatuses have been described in the foregoing, various modification are possible and are intended to be included within the scope of the present disclosure.

Although the systems, methods, and apparatuses described above disclosure are for use with those who do not require breathing assistance, in some embodiments the systems and apparatuses, or portions thereof, can be used in combination with a respirator or ventilator to deliver medications in purified air to patients with breathing difficulties. Examples of personal respirators are those described in U.S. patent application Ser. No. 11/552,871 entitled “Methods and Systems of Delivering Medication Via Inhalation” and U.S. patent application Ser. No. 11/533,529 entitled “Respirators for Delivering Clean Air to an Individual User,” which are both hereby incorporated by reference into the present disclosure.

Although air filtration has been described in detail in the foregoing, pure air can alternatively be synthesized, such as by mixing the gases from reservoirs of liquid oxygen, liquid nitrogen, and liquid carbon dioxide.

Although the systems, methods, and apparatuses of the present disclosure have been described above with respect to a delivery system employing a user interface in the form of a mask, an interface can alternatively comprise an intubation tube of an intubated patient such that medicine is delivered directly into the trachea.

It is further noted that operation of the components that control droplet evaporation can be automated in embodiments in which the system includes one or more sensors that collect information about the operating environment (e.g., temperature and/or humidity) and provide that information to an appropriate control component. In such embodiments, components of the system, such as droplet size control elements, can be automatically controlled to achieve optimal droplet evaporation and size reduction for substantially any operating environment.

In the above disclosure, various actions are described to ensure that the medicine droplets have the right size for deposition within the alveoli, for example, approximately 1 to 3 μm. That does not necessarily mean, however, that the droplets must have a diameters in that range upon entry into the user's respiratory tract given that the droplets may hygroscopically increase in size within the respiratory tract (e.g., lungs). Studies have shown that such hygroscopic growth of small droplets can be significant over very short time intervals, including intervals of time between generation of the droplets and their final deposition in the lung. For example, particles may increase in size by a factor of 2 or 3 within the respiratory tract. Therefore, the provision of very small, even sub-micron, droplet diameters to the respiratory tract may be preferable. For example, in some embodiments, droplets having diameters of approximately 0.5 to 1.5 μm may be preferable. In that hygroscopic growth is also dependent upon droplet composition, the preferred droplet size may vary depending upon the nature of the medication being administered.

Another aspect of the disclosed systems, methods, and apparatuses is the ability to accurately monitor the pressure and flow parameters of the filtered and medicated air being supplied to the user. Electronic sensors can be used to actively monitor and respond to the respiratory cycle of the user. For example, an array of solid state pressure transducers, such as the SM5600 series sensors produced by Silicon Microstructures of Milipitas, Calif., can be used to monitor the pressure conditions within the drug delivery unit. Data from the sensors are monitored in real-time by an on-board microprocessor that stores the data collected from the sensors. Through analysis of this data the processor can establish or “learn” baseline respiratory parameters of the user based on approximately one or two minutes worth of data. Once baseline parameters are established the processor may react appropriately to the user's unique requirements and breathing patterns. As one example, the processor may observe pressure readings to detect a particularly rapid or deep (large volume) inhale cycle at its onset. In this manner the processor may cause the drug delivery device to inject a precisely-controlled amount of medicine in the airstream at precisely the correct time for it to be most deeply and effectively inhaled by the user. In another case, the drug delivery device, as controlled by the processor, may administer drugs only during alternate inhalations.

Furthermore, it is noted that the disclosed systems, methods, and apparatuses can include appropriate sensors that provided feedback that is useful in controlling droplet size. For example, the system can include sensors that monitor one or more of current atmospheric temperature, humidity, and pressure to provide the system with an indication as to whether droplet size adjustment is necessary and, if so, to what extent. In such a case, the monitored condition(s) can be used to reference a look-up table or to execute an algorithm that indicates what actions should be taken, if any. As a further example, the system can include a sensor that detects the size of droplets just before they exit the system to provide an indication as to whether droplet size adjustment is warranted. In such a case, the detected droplet size can be used to reference a look-up table or to execute an algorithm that indicates what actions should be taken, if any.

The processor may receive input from “smart” drug cartridges in a manner similar to the way ink jet printers for personal computers receive data from ink jet cartridges. This data may be used to instruct the processor regarding the optimal parameters for delivery for the drug and the patient as determined by a doctor of pharmacist. Such data might include information on dosages, proper timing of the dose with the user's respiratory cycle, etc.

In further embodiments, the drug delivery device can include a data port which may be connected to a device for delivering feedback on the user's condition.

We claim:

1. A portable system for pulmonary drug delivery, the system comprising:
a portable air supply unit comprising an air mover configured to generate a positive pressure airflow;
a drug delivery unit configured to inject droplets of medication into the airflow generated by the air supply unit; and
a user interface configured to deliver the airflow and droplets to a user.

2. The system of claim 1, wherein the portable air supply unit is configured to be carried or worn by the user.

3. The system of claim 1, wherein the air mover is configured to generate airflow of approximately 50 to 500 slm such that the airflow is low enough so as not to disturb the user's normal breathing patterns.

4. The system of claim 1, wherein the portable air supply unit further comprises a particle filter configured to purify the airflow generated by the air mover.

5. The system of claim 4, wherein the particle filter is configured to filter out particles having a size of 10 nanometers or more.

6. The system of claim 4, wherein the particle filter has a usable surface area of approximately 2700 cm³ to 5400 cm³.

7. The system of claim 1, wherein the drug delivery unit comprises a body portion that defines an inner passage through which the generated airflow flows, a medication containment element configured to hold medication to be injected into the airflow, and a droplet ejection device configured to inject the medication held by the medication containment element into the inner passage.

8. The system of claim 7, wherein the droplet ejection device comprises an ejection head that includes droplet ejection elements that are activated to generate the droplets.

9. The system of claim 8, wherein the droplet ejection elements comprise heater resistors.

10. The system of claim 1, wherein the user interface comprises a face mask configured to fit over the user's nose and mouth.

11. The system of claim 1, further comprising a supply hose that extends between the drug delivery unit and the user interface.

12. The system of claim 10, further comprising a second supply hose that extends between the portable air supply unit and the drug delivery unit.

13. The system of claim 1, further comprising a medication heating element configured to heat the medication before it is injected into the airflow.

14. The system of claim 1, further comprising an air heating element configured to heat the airflow before it reaches the user.

15. The system of claim 1, further comprising a humidity control element configured to adjust the humidity of the airflow before it reaches the user.

16. The system of claim 1, further comprising a turbulence element configured to increase turbulence of the airflow.

17. The system of claim 1, further comprising a monitoring unit configured to collect patient data and respiration data measured by the drug delivery unit.

18. The system of claim 17, wherein the monitoring unit is further configured to transmit the collected data to another component.

19. A portable system for pulmonary drug delivery, the system comprising:
a portable air supply unit configured to be carried or worn by a user, the unit comprising a housing that defines an interior space in which is provided a fan configured to generate a positive pressure airflow and a particle filter configured to remove particulate matter from the airflow to purify the airflow;
a first supply hose extending from the portable air supply unit configured to receive the generated airflow;
a drug delivery unit connected to the first supply hose, the drug delivery unit comprising a medicine containment unit configured to hold medicine and a droplet ejection device that includes heater resistors configured to eject droplets of the medication into the generated airflow;
a second supply hose extending from the drug delivery unit configured to receive the generated airflow and ejected droplets of medication; and
a face mask connected to the second supply hose configured to surround the user's nose and mouth and deliver the airflow and droplets to the user's respiratory system.

20. The system of claim 19, wherein the fan is configured to generate airflow of approximately 50 to 500 slm such that the airflow is low enough so as not to disturb the user's normal breathing patterns.

21. The system of claim 19, wherein the particle filter is configured to filter out particles having a size of 10 nanometers or more.

22. The system of claim 19, further comprising a medication heating element configured to heat the medication before it is injected into the airflow.

23. The system of claim 19, further comprising an air heating element configured to heat the airflow before it reaches the user.

24. The system of claim 19, further comprising a humidity control element configured to adjust the humidity of the airflow before it reaches the user.

25. The system of claim 19, further comprising a turbulence element configured to increase turbulence of the airflow.
